(19) United States
(12) Patent Application Publication
(10) Pub. No.: US 2013/0267823 A1
(43) Pub. Date: Oct. 10, 2013

(54) MEASURING APPARATUS

(71) Applicants: Takao Nakajima, Kyoto-shi (JP); Yasufumi Asao, Kyoto-shi (JP)

(72) Inventors: Takao Nakajima, Kyoto-shi (JP); Yasufumi Asao, Kyoto-shi (JP)

(73) Assignee: CANON KABUSHIKI KAISHA, Tokyo (JP)

(21) Appl. No.: 13/993,730

(22) PCT Filed: Dec. 28, 2011
(86) PCT No.: PCT/JP2011/080602

§ 371 Date: Jun. 13, 2013

(30) Foreign Application Priority Data
Jan. 7, 2011 (JP) 2011-001888
Dec. 7, 2011 (JP) 2011-267794

(51) Int. Cl. A61B 5/00

(52) U.S. Cl. CPC A61B 5/0095; A61B 5/708; A61B 5/72; A61B 5/742
USPC 600/407

(57) ABSTRACT

A measuring apparatus has a holding unit for holding an object, and a probe including a receiving element for receiving through the holding unit an acoustic wave generated by the object irradiated with light. The light is applied to an object surface held by the holding unit. The probe is arranged such that a direction of a normal to the object surface held by the holding unit is nonparallel to a direction in which the receiving element exhibits its highest reception sensitivity.

### TECHNICAL FIELD

This invention relates to a measuring apparatus.

### BACKGROUND ART

In general, imaging apparatuses employing X-rays, supersonic waves, or MRI (Magnetic Resonance Imaging) are commonly used in the medical field. On the other hand, researches have been actively conducted in the medical field for the purpose of realizing an optical imaging apparatuses which are designed to acquire information in a living body by causing light, such as a laser beam, emitted from a light source to propagate within an object such as a living body, and detecting the propagated light. Photoacoustic Tomography (PAT) is proposed as one of such optical imaging technologies (NPL 1: Non-patent literature 1).

According to the PAT technology, pulsed light generated by alight source is applied to an object so that optical energy is propagated and diffused in the object, and acoustic waves generated by body tissues absorbing this optical energy (hereafter, referred to as photoacoustic waves) are detected at a plurality of spots in the body. Subsequently, signals thus obtained are analyzed so that information relating to optical property values within the object is visualized. This makes it possible to obtain an optical property value distribution, particularly optical energy absorption density distribution within the object.

In the photoacoustic tomography, according to Non-patent literature 1, an initial acoustic pressure (P₀) of photoacoustic waves generated by an absorber of the object as a result of absorption of light can be represented by the following expression (1).

P₀=Γ·μₐ·Φ (1)

In this expression, Γ denotes a Gruneisen coefficient, which is obtained by dividing a product of a coefficient of cubic expansion (β) and a square of sonic speed (c) by a specific heat at constant pressure (C_{P}). μₐ denotes an optical absorption coefficient of an absorber, and Φ denotes an amount of light in a local region (amount of light applied to the absorber, also referred to as optical fluence).

Many cases have been reported in which a blood vessel in a living body is imaged with PAT by utilizing the fact that hemoglobin in the blood absorbs a large amount of light in the living body.

In recent years, as reported in NPL 2 (Non-patent literature 2), researches have been conducted to use PAT for detecting breast cancer. Breast cancer induces angiogenesis around a tumor in its process of growth. It is believed this is the reason why a larger amount of light is absorbed in the region around the tumor than in surrounding adipose tissues or the like.

In photoacoustic imaging, as shown in the expression (1), the acoustic pressure of acoustic waves obtained from the absorber in the living body by light absorption is proportional to the local amount of light reaching the absorber.

Light applied to the living body is rapidly attenuated by scattering and absorption in the body. Therefore, the acoustic pressure of the acoustic waves generated in the tissues deep in the body is significantly attenuated in accordance with the distance from the light irradiation position. Furthermore, there is a limit to the amount of light that can be applied to the living body. Therefore, it is difficult to use PAT to perform imaging of a deep region of the living body.

There arises a similar problem when imaging of breast cancer is performed by using PAT. One of the effective solutions for this problem is to compress the bread to reduce the thickness thereof, as is often used in X-ray mammography. FIG. 1 shows a configuration of such an apparatus as disclosed in Non-patent literature 2. In the apparatus shown in FIG. 1, the breast 12 is sandwiched and compressed between a glass plate 10 and a probe 11, and light 13 is applied from the glass plate side.

### CITATION LIST

### Non Patent Literature

NPL 1: M. Xu, L. V. Wang, “Photoacoustic imaging in biomedicine”, Review of scientific instruments, 77, 041101 (2006)
NPL 2: S. Manohar et al., “Region-of-interest breast studies using the Twente Photoacoustic Mammoscope (PAM)”, Proc. Of SPIE Vol. 6437 643702-1

### SUMMARY OF INVENTION

However, when the compressed breast is irradiated with light from the side opposite the probe (hereafter, referred to as the “non-probe side irradiation”) as described in Non-patent literature 2, the light reaching the side of the breast close to the probe will be very weak, and it is difficult to image any cancer (absorber) present at such a position. It may be possible to image the tumor present on the side close to the probe by compressing the breast as much as possible. However, it is not preferable to raise the compression pressure, possibly resulting in increased burden and pain given to the patient.

One of the methods to enable imaging of a cancer present in the side of the breast close to the probe without raising the amount of compression is to apply light from the side of the probe as well (hereafter, referred to as the “probe side irradiation”). With this method, a sufficient amount light reaches the region of the breast on the side of the probe as well, whereby the probe is enabled to detect a photoacoustic signal generated by a cancer present in the side close to the probe.

However, when the probe-side irradiation is performed, a large photoacoustic signal is generated at the surface of the living body irradiated with the light. This is because while the acoustic pressure of photoacoustic waves is proportional to the local amount of light reaching the absorber, as indicated by the expression (1), the light is not attenuated at the surface of the living body and hence the amount of light is large. This large photoacoustic signal from the surface of the living body will cause an artifact in a reconstructed image and the image will be deteriorated significantly. Furthermore, if a photoacoustic signal from a tumor to be detected is obscured by this large photoacoustic signal from the surface of the living body, the cancer cannot be detected.

Still further, if there is a member for compressing the object between the probe and the object, the photoacoustic waves will be multiply-reflected in the inside of the member. This multiply-reflected signal will overlap with a signal generated in a deeper region as viewed from the probe, and the image of the deep region will also be deteriorated. Since the photoacoustic signal from a tumor present in a deep region is small, the accuracy of detecting a cancer in a deep region is deteriorated consequently.

As described above, when the object is compressed, the probe-side irradiation is required to perform imaging at every depth. However, the probe-side irradiation will induce a problem of deterioration of the image quality and adverse effects on imaging of an optical absorber (e.g. breast cancer) within a living body.

This invention has been made in view of the problems described above, and it is an object of the invention to provide a technique for performing imaging of object information over a wide range in a depth direction of the object while suppressing image deterioration.

In order to achieve the object, this invention provides

a measuring apparatus comprising:

a holding unit for holding an object; and

a probe including a receiving element for receiving, through the holding unit, an acoustic wave generated by the object irradiated with light, wherein

the light is applied to an object surface held by the holding unit; and

the probe is arranged such that a direction of a normal to the object surface held by the holding unit is nonparallel to a direction in which the receiving element exhibits the highest reception sensitivity.

According to this invention, it is made possible to perform imaging of object information over a wide range in a depth direction of the object while suppressing image deterioration.

Further features of the present invention will become apparent from the following description of exemplary embodiments with reference to the attached drawings.

### BRIEF DESCRIPTION OF DRAWINGS

FIG. 1 is a diagram illustrating a configuration example of an object information imaging apparatus holding an object;

FIG. 2 is a diagram illustrating a model for explaining a principle of this invention;

FIG. 3 is another diagram illustrating a model for explaining the principle of this invention;

FIG. 4 is another diagram illustrating a model for explaining the principle of this invention;

FIG. 5 is a diagram illustrating a configuration example of a measuring apparatus to which this invention is applicable;

FIG. 6 is a diagram illustrating another configuration example of a measuring apparatus to which this invention is applicable;

FIG. 7 is a diagram illustrating another configuration example of a measuring apparatus to which this invention is applicable;

FIG. 8 is a diagram illustrating an example of an arrangement used in an experiment conducted for verifying the principle of this invention;

FIG. 9A is a diagram illustrating a result of the verification experiment for the principle of this invention;

FIG. 9B is a graph illustrating the result of the verification experiment for the principle of this invention;

FIG. 10A is a diagram illustrating a distance between the optical absorber and normal receiving surface; and

FIG. 10B is a diagram illustrating a distance between the optical absorber and inclined receiving surface.

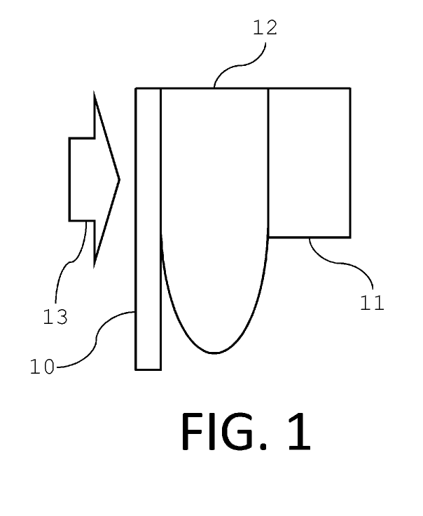

### DESCRIPTION OF EMBODIMENTS

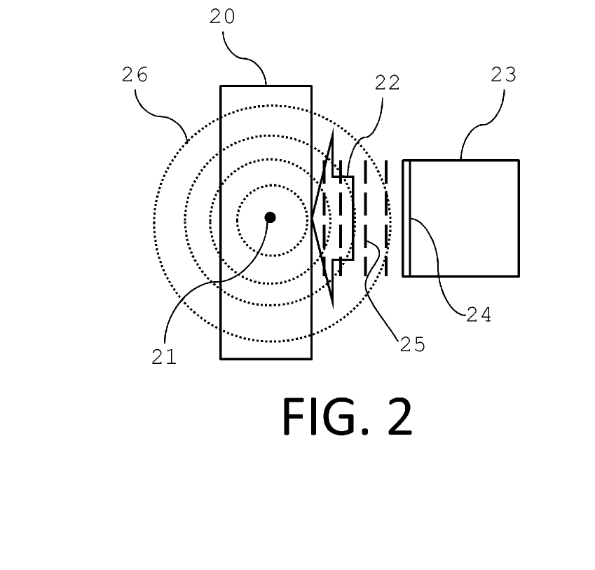

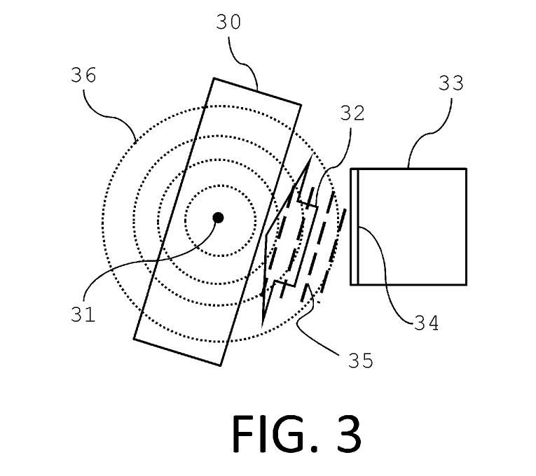

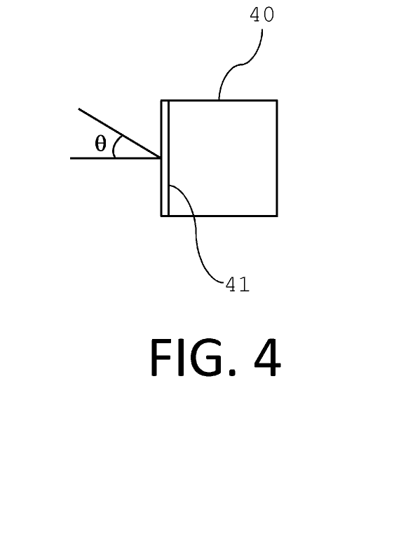

A principle of this invention will be described, wherein an arrangement of a measuring apparatus (object information imaging apparatus) according to the invention makes it possible to suppress deterioration of image when performing probe-side irradiation. FIG. 2 and FIG. 3 show a model for explaining the principle. FIG. 4 is a diagram showing an angle θ relative to a receiving element surface 41 of a probe 40.

In FIG. 2, an object 20 has a shape of rectangular parallelepiped, mimicking a breast which is held in a planar shape. There exists in the object 20 a spherical optical absorber 21 mimicking a cancer. Light 22 is applied to an area that is sufficiently wider than a reaching distance of light within the living body, so that the density of amount of light applied to the object 20 becomes uniform. The light irradiation surface of the object 20 is arranged parallel to the receiving element surface 24 of the probe 23.

Photoacoustic waves generated by this irradiation of light 22 from the light irradiation surface of the planar shaped object 20 become plane waves 25. The photoacoustic waves become plane principally under conditions that the density of amount of irradiated light is uniform, and the light is applied to an area that is wider than a reaching distance of the light within the living body. The plane waves 25 are propagated in a direction toward the probe 23, and incident perpendicularly to the receiving element surface 24 of the probe 23. On the other hand, photoacoustic waves generated by the spherical optical absorber 21 become spherical waves 26 propagated concentrically. In general, the direction in which the highest reception sensitivity of the receiving element can be achieved is a perpendicular direction to the element surface, as described later.

In FIG. 3, the light irradiation surface of an object 30 is not arranged parallel to a receiving element surface 34 of a probe 33, but is arranged at a tilt angle θ. This means that the probe is arranged such that a direction of a normal to the light irradiation surface is nonparallel to the direction in which the highest reception sensitivity of the receiving element is achieved. Other than this, the configuration is the same as that of FIG. 2.

In this configuration, photoacoustic waves generated from the light irradiation surface by irradiation of light 32 are plane waves 35. The plane waves 35 are propagated in a direction tilted in an angle θ relative to the probe 33, and are incident to the receiving element surface 34 of the probe at the tilt angle θ. Meanwhile, photoacoustic waves generated by a spherical optical absorber 31 become spherical waves 36 propagated concentrically in the same manner as the arrangement of FIG. 2.

Description will be made, with reference to FIG. 4, of relationship between incident direction of supersonic waves and reception sensitivity (directivity of reception sensitivity) of a supersonic probe. When the receiving element is of a circular shape, its reception sensitivity d(θ) is represented by the following expression (2).

$$\begin{matrix}[ Math . 1 ] & \\ d ( θ ) = 2 \cdot \vert \frac{{J}_{1} ( k \cdot a \cdot sin ( θ ) )}{k \cdot a \cdot sin ( θ )} \vert & ( 2 )\end{matrix}$$

In this expression (2), θ denotes an incident angle, a denotes a radius of the receiving element, k denotes an angular frequency of supersonic waves, and J₁ denotes a Bessel function.

When the receiving element is of a rectangular shape, its reception sensitivity is represented by the following expression (3).

$$\begin{matrix}[ Math . 2 ] & \\ d ( θ ) = 2 \cdot \vert \frac{sin ( k \cdot a \cdot sin ( θ ) )}{k \cdot a \cdot sin ( θ )} \vert & ( 3 )\end{matrix}$$

In this expression (3), a denotes a length of a side of the receiving element.

These expressions reveal that as the incident angle θ becomes greater, that is, as the incident direction is more inclined, the reception sensitivity becomes lower. They also reveal that the dependency of the reception sensitivity on the incident angle varies depending on size of the receiving element or frequency of the supersonic waves. In general, the directivity is increased (enhanced) when the receiving element is large in size, or when the frequency of supersonic waves received by the receiving element is high. This means that, in these cases, the reception sensitivity is reduced as the incident direction is more inclined.

Therefore, it is preferable that the angle between the surface of the object and the surface of the receiving element of the probe is adjusted according to the directivity of the reception sensitivity of the receiving element. Further, it is preferable that the angle between the surface of the object and the surface of the receiving element of the probe is adjusted according to an element size of the receiving element or a reception frequency.

The plane waves 25 generated in the arrangement of FIG. 2 are incident on the receiving element surface 24 perpendicularly, that is, θ is zero degrees. Therefore, the plane waves 25 can be received with high sensitivity. In contrast, the plane waves 35 generated in the arrangement of FIG. 3 are incident on the receiving element surface 34 at an angle θ. Therefore, the plane waves 35 are received with reduced sensitivity in accordance with the magnitude of the incident angle θ. Due to such directivity of the reception sensitivity, the probe in FIG. 3 receives smaller plane waves than the probe in FIG. 2 does, even if plane photoacoustic waves with the same intensity are generated at the surface of the object in FIG. 2 and FIG. 3.

On the other hand, the photoacoustic waves 26 generated by the spherical optical absorber 21 in FIG. 2 and the photoacoustic waves 36 generated by the spherical optical absorber 31 in FIG. 3 are both spherical waves. Therefore both the photoacoustic waves 26 and 36 are incident on the receiving element of the probe at the same incident angle. Therefore, when photoacoustic waves of the same intensity are generated by the spherical optical absorbers in FIG. 2 and FIG. 3, respectively, the sensitivity of the probes to the spherical waves become the same in FIG. 2 and FIG. 3.

This means that when the arrangement shown in FIG. 3 is employed, only the sensitivity to the plane waves generated at the light irradiation surface object 20 can be reduced more than the arrangement shown in FIG. 2 without changing the sensitivity to the spherical waves. In other words, when the light irradiation surface of the object 30 is inclined at a certain angle θ relative to the receiving element surface 34 of the probe 33 and the both surfaces are irradiated with light in the same manner, the photoacoustic signals that the probe receives from the object surface can be reduced in contrast to the photoacoustic signals from the spherical absorber.

The angle of the receiving element surface of the probe relative to the surface of the object is preferably such an angle at which the reception sensitivity of the receiving element of the probe is equal to or less than one fourth of the maximum value thereof and the magnitude of the reception sensitivity assumes a value of zero or more.

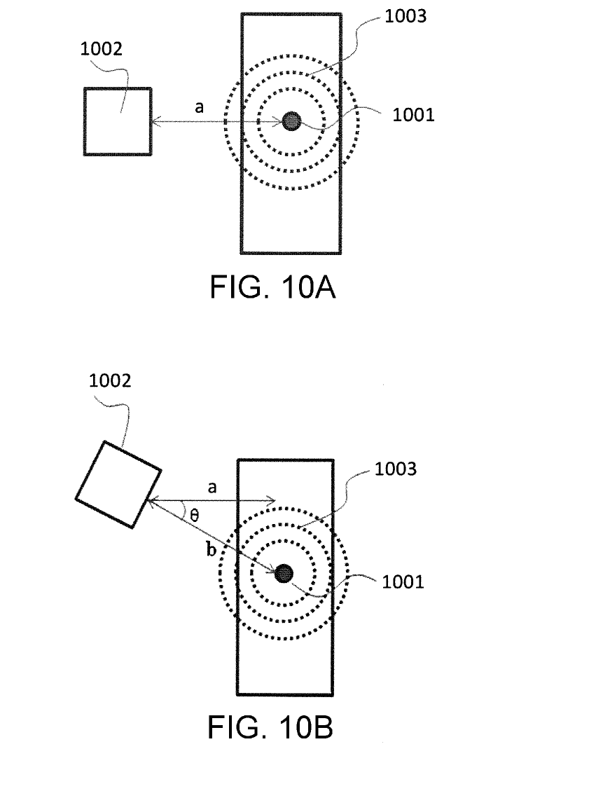

On the other hand, the receiving intensity when spherical wave from the spherical optical absorber is received by the probe will be described according to FIGS. 10A and 10B.

In FIG. 10, the acoustic pressure of the spherical wave 1003, generated from the spherical optical absorber 1001 and propagates in spherical shape, decreases in inverse proportion to the distance between the probe and the spherical optical absorber 1001.

As shown in FIG. 10A, when θ=0, the distance between the spherical optical absorber 1001 and receiving surface of the probe 1002 is a. On the other hand, as shown in FIG. 10B, when θ≠0, the distance between the spherical optical absorber 1001 and receiving surface of the probe 1002 is b (b=a/cos θ). The acoustic pressure of the photoacoustic wave (spherical wave) 1003 which propagates in spherical shape is in inverse proportional to the distance between the probe and the spherical optical absorber 1001, and therefore the received acoustic pressure (intensity) at the probe 1002 decreases in proportion to cos θ.

As remarked above, when the probe is inclined at θ degree, the spherical wave to be observed also decreases. That means, if θ is overly increased to reduce the plane wave, the spherical wave, which is primary observation object, becomes hard to find.

In light of this knowledge, more preferably, the angle of the receiving element surface of the probe relative to the surface of the object is such an angle at which the reception sensitivity assumes a value equal to or less than one fourth but not less than one 100th of the maximum value thereof. This configuration makes it possible to make further smaller the reception signal of the plane waves generated at the light irradiation surface of the object 20.

Further, the angle of the receiving element surface of the probe relative to the object surface is preferably equal to or more than 10 degrees but not more than 80 degrees, more preferably equal to or more than 10 degrees but not more than 60 degrees, and optimally equal to or more than 20 degrees but not more than 50 degrees.

As a result, when an image is reconstructed using the photoacoustic signal obtained in the arrangement shown in FIG. 3, deterioration of the image caused by the photoacoustic signal generated at the object surface can be suppressed more in comparison with when the image is reconstructed using the photoacoustic signal obtained in the arrangement shown in FIG. 2. This makes it possible to perform high-definition imaging of an optical absorber such as a cancer present in the inside of the object.

In addition to the directivity of reception sensitivity, the arrangement of FIG. 3 can be employed, in which the traveling direction of the plane waves from the object surface is inclined, so that the plane waves are deflected from the probe to inhibit the reception of the plane waves.

Exemplary embodiments of this invention will be further described with reference to the accompanying drawings. The following description will be made, taking a living body information imaging apparatus as an example, in which an object information imaging apparatus according to this invention is applied to a living body. However, an object to be measured according to the invention is not limited to this.

### First Embodiment

Firstly, a description will be made of a living body information imaging apparatus according to a first embodiment of this invention.

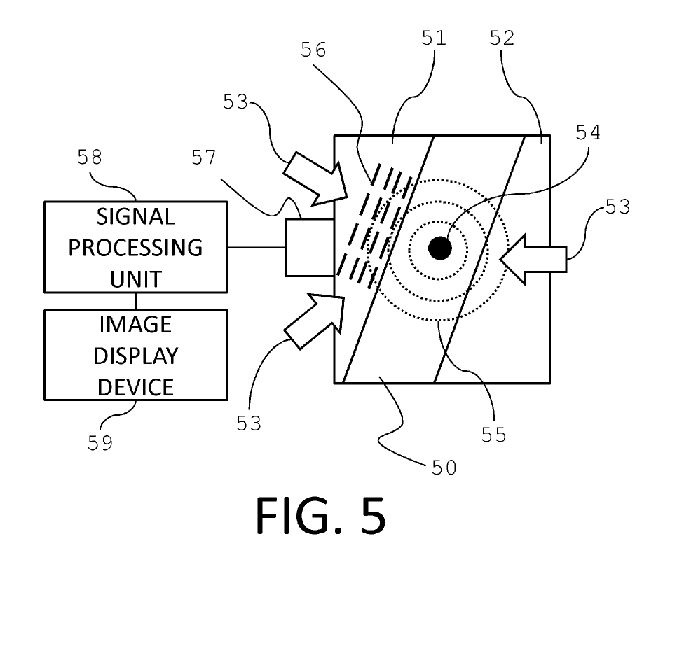

FIG. 5 is a diagram illustrating a configuration example of the living body information imaging apparatus according to the first embodiment. The living body information imaging apparatus according to this embodiment is configured to enable imaging of distribution of optical property values in a living body and distribution of density of substances forming body tissues obtained based on such information for the purpose of diagnosis of a tumor or vascular disease, or a follow-up thereof.

The living body information imaging apparatus according to this embodiment has holding units 51 and 52 for holding a living body 50. The living body 50 thus held is irradiated with irradiation light 53.

The living body information imaging apparatus further has a probe 57. The probe 57 detects a photoacoustic wave 55 generated by a tumor, a blood vessel or such other optical absorber 54 present in the living body absorbing part of the optical energy, or a photoacoustic wave 56 generated at a surface of the living body, and converts the detected photoacoustic wave into an electric signal.

The living body information imaging apparatus further has a signal processing unit 58 which analyzes the electric signal to generate image data which serves as original data such as information on optical property value distribution, for displaying an image for the user. The image display device 59 also displays a result of processing by the signal processing unit.

The holding units 51 and 52 are formed by a pair of plate-like members having opposing faces inclined. Two such members are used to compress and hold the living body 50 sandwiched between them. Thus, the sides of the living body 50 facing the holding units 51 and 52 are made flat. One of the pair of planes of the holding unit 51 serves as a holding face for holding the living body 50, while the probe 57 is arranged on the other. The holding unit 51 is preferably made of a material which is highly optically transmissive and has high durability against light. More preferably, the holding unit 51 is made of a material in which attenuation of acoustic waves is small and which exhibits an acoustic impedance similar to that of the living body. Such a material may be exemplified by polymethylpentene.

An acoustic matching medium is desirably provided between the holding unit 51 and the living body 50, and between the holding unit 51 and the probe 57, for suppressing reflection of acoustic waves. For example, an impedance matching gel or the like may be used as the medium.

The holding unit 52 is preferably made of a material which is highly optically transmissive and has high durability against light. For example, glass or acrylic may be used as such a material.

Hereafter, the side of the living body 50 facing the holding unit 51 on which the probe 57 is arranged shall be referred to as the “probe side”, and the side of the living body 50 facing the holding unit 52 shall be referred to as the “non-probe side”.

The irradiation light 53 used herein is light having such wavelength characteristics that the light is absorbed by specific components of the components forming the living body 50. Although the irradiation light 53 is applied to both of the probe side and the non-probe side in this embodiment, the irradiation light 53 may be applied only to the probe side. Further, although the irradiation to the probe side is performed from both sides of the probe, this is not always necessary as long as light is applied to the surface of the living body 50 located in front of the probe 57. For example, the light may be applied only from one side of the probe.

The irradiation light 53 is preferably applied with an amplitude (size, diameter) greater than a reaching distance of the irradiation light in the living body (object). For example, when an effective attenuation coefficient of light is denoted by μ_{eff}, the irradiation light 53 is preferably applied with an amplitude greater than 1/μ_{eff}. The effective attenuation coefficient μ_{eff} can be expressed by the following expression (4).

[Math. 3]

μ_{eff}=\sqrt{3·μₐ·μₛ′} (4)

In this expression (4), μa denotes an absorption coefficient of light, and μs′ denotes an equivalent scattering coefficient.

The irradiation light 53 is preferably applied such that the distribution of irradiation light amount density becomes uniform. In order to uniformize the distribution of irradiation light amount density, a diffuser or a fly-eye lens can be used.

Pulsed light may be used as the irradiation light 53. The pulsed light preferably is of on the order of several nano seconds to several hundreds of nano seconds, and preferably has a wavelength of 400 nm or more but not more than 1600 nm.

Although a laser is preferred as the light source for generating the irradiation light 53, a light emitting diode or the like may be used in place of the laser. Various types of lasers can be used as the laser, such as a solid laser, a gas laser, a dye laser, and a semiconductor laser.

By using a dye laser or an OPO (Optical Parametric Oscillator) oscillating at a wavelength which can be converted, it is also made possible to measure a difference of the optical property value distribution according to the wavelength.

The light source used here preferably has a wavelength in the range from 700 nm to 1100 nm, since light with such a wavelength is absorbed little in the living body. However, it is also possible to use a wavelength range wider than this, such as a wavelength range from 400 nm to 1600 nm, or even a terahertz wavelength range, microwave wavelength range, and radiowave wavelength range.

Further, the light source of the irradiation light 53 can be configured to scan the surface of the living body 50.

The probe 57 detects acoustic waves (which are typically supersonic waves, and also referred to as photoacoustic waves) generated in the living body by partial absorption of the energy of the irradiation light 53, and converts the detected acoustic waves into an electric signal.

The probe may be any type of acoustic wave detector as long as it can detect an acoustic wave signal, such as a transducer utilizing a piezoelectric phenomenon, a transducer utilizing optical resonance, and a transducer utilizing change in capacitance. Like the light source 53, the probe 57 also may be configured to scan the surface of the object 50.

Although this embodiment relates to a case in which an array-type probe 57 in which receiving elements are arranged in a two-dimensional array is provided, the invention is not limited to such an arrangement, but any other arrangement may be employed as long as the acoustic waves can be detected at a plurality of places. Since the same effect can be obtained as long as the acoustic waves are detected at a plurality of places, a probe with a single receiving element (single transducer) may be configured to scan the surface of the holding unit 51.

If the electric signal obtained by the probe 57 is small, it is preferable to amplify the signal intensity with an amplifier.

The signal processing unit 58 according to this embodiment calculates, based on the electric signals obtained from the probe 57, a position and magnitude of the absorber 54 in the living body, or an optical property value distribution such as a distribution of amounts of optical energy accumulation or optical absorption coefficients.

Universal back-projection or phasing addition is conceivable as a reconstruction algorithm for obtaining an optical property value distribution based on the electric signal obtained at the plurality of places. According to this embodiment, it is required to take into consideration, in using any of these algorithms, refraction of the acoustic waves or change of the sonic speed caused by the holding unit 51 located between the living body 50 and the probe 57, and the angle of the receiving element surface relative to the object surface.

Any type of processing unit may be used as the signal processing unit 58 as long as it is able to store an intensity of acoustic waves and its time variation, and convert them into optical property value distribution data by means of computing means. For example, an oscilloscope and a computer capable of analyzing data stored in the oscilloscope may be used.

When light with a plurality of wavelengths is used, an optical coefficient in the living body is calculated for each of the wavelengths, and the values thus obtained are compared with a unique wavelength dependency of a substance forming the body tissues (glucose, collagen, oxygenated or reduced hemoglobin, or the like). This also makes it possible to image density distribution of the substance forming the living body.

In this embodiment of the invention, an image display device 59 is desirably provided to display image information obtained by the signal processing.

The use of the living body information imaging apparatus as described in this embodiment makes it possible to perform imaging of object information over a wide range in a depth direction in the object while suppressing deterioration of the image.

### Second Embodiment

A living body information imaging apparatus according to a second embodiment of this invention will be described.

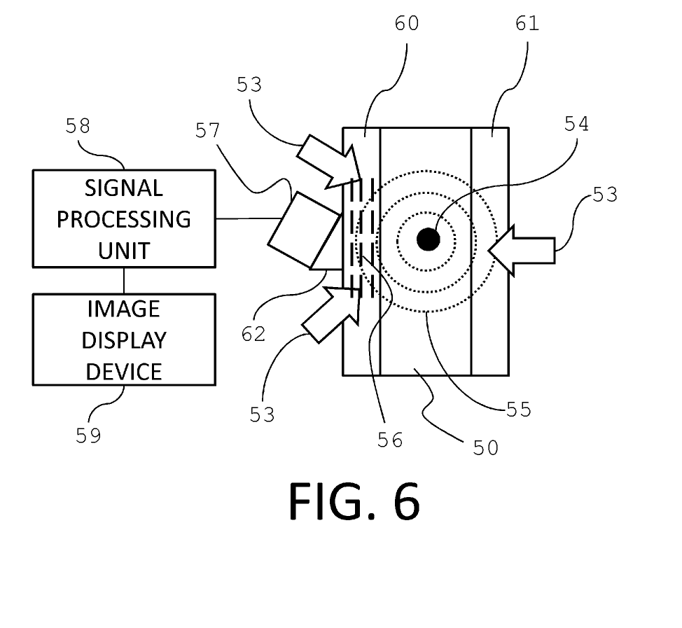

FIG. 6 is a diagram illustrating a configuration example of a living body information imaging apparatus according to this embodiment. Those components common with the apparatus shown in FIG. 5 are denoted by the same reference numerals and detailed description thereof will be omitted.

The living body information imaging apparatus according to this second embodiment has holding units 60 and 61 for holding a living body 50. The living body 50 thus held is irradiated with irradiation light 53.

The living body information imaging apparatus further has a probe 57. The probe 57 detects photoacoustic waves 55 generated by an optical absorber 54 present in the living body, such as a tumor, a blood vessel or the like, absorbing part of the optical energy, or photoacoustic waves 56 generated at a surface of the living body, and converts the detected photoacoustic waves into an electric signal.

The living body information imaging apparatus according to this embodiment has a member 62 arranged between the living body 50 and the probe 57, the member 62 having a pair of planes forming an angle therebetween.

The living body information imaging apparatus further has a signal processing unit 58 for acquiring optical property value distribution information by analyzing the electric signal. An image display device 59 is provided to display a result of the processing performed by the signal processing unit.

According to this embodiment, the holding units 60 and 61 are formed of flat plate-like members arranged in parallel to each other. Two such members are used to hold the living body 50 sandwiched between them. The holding unit 60 is preferably made of a material which is highly optically transmissive and has high durability against light. More preferably, the holding unit 1 is made of a material in which attenuation of acoustic waves is small and which exhibits an acoustic impedance similar to that of the living body. Polymethylpentene for example may be used as such a material. The holding unit 60 holds the living body 50 at one of the pair of planes, and a probe 57 is arranged on the other plane.

There is arranged, between the living body 50 and the probe 57, a member 62 having a pair of planes forming an angle therebetween. The member 62 is preferably made of a material in which attenuation of acoustic waves is small, which has an acoustic impedance similar to that of the living body. Polymethylpentene or acrylic, for example, may be used as such a material.

It is desirable to provide an acoustic matching medium between the holding unit 60 and the living body 50, and between the holding unit 60 and the member 62, and further between the probe 57 and the member 62, in order to suppress reflection of acoustic waves. For example, impedance matching gel may be used as the acoustic matching medium.

The holding unit 61 is preferably made of a material which is highly optically transmissive and has high durability against light. For example, glass or acrylic may be used as such a material.

In this embodiment, the irradiation light, the probe, the signal processing unit, and the image display device may be the same as those of the first embodiment.

The use of the living body information imaging apparatus as described in this embodiment makes it possible to perform imaging of object information over a wide range in a depth direction in the object while suppressing deterioration of the image.

### Third Embodiment

A living body information imaging apparatus according to a third embodiment of this invention will be described.

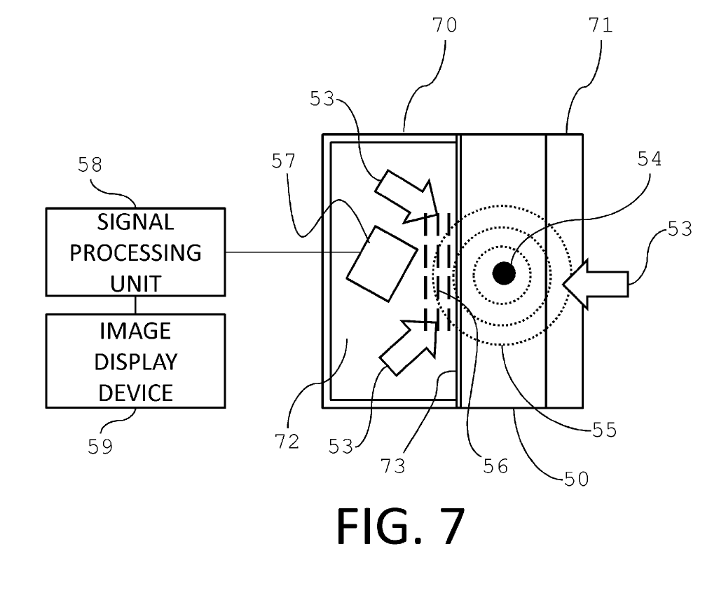

FIG. 7 illustrates a configuration example of a living body information imaging apparatus according to this embodiment. Those components common with the apparatus shown in FIG. 5 are denoted by the same reference numerals and detailed description thereof will be omitted.

The living body information imaging apparatus according to this embodiment has holding units 70 and 71 for holding a living body 50. The living body 50 thus held is irradiated with irradiation light 53.

The living body information imaging apparatus further has a probe 57 which detects photoacoustic waves 55 generated by a tumor, a blood vessel, or such other optical absorber 54 present in the living body absorbing part of the optical energy, or photoacoustic waves 56 generated at a surface of the living body, and converts the detected photoacoustic waves into an electric signal.

The living body information imaging apparatus further has a signal processing unit 58 which analyzes the electric signal to obtain optical property value distribution information, and an image display device 59 displaying a result of the processing.

The holding unit 70 and 71 holds the living body 50 sandwiched therebetween. The holding unit 70 has a container-like shape, and the living body 50 is held on the bottom face 73 of the holding unit 70 so as to assume a planar shape. The interior of the container is filled with an acoustic matching medium 72. Water or castor oil can be used as the acoustic matching medium 72. The probe 57 is arranged within the acoustic matching medium such that the reception face of the probe is inclined with respect to the surface of the held living body.

The holding unit 70 is preferably made of a material which is highly optically transmissive and has high durability against light. More preferably, the holding unit 70 is made of a material in which attenuation of acoustic waves is small and which exhibits an acoustic impedance similar to that of the living body. Polymethylpentene for example may be used as such a material. The bottom face 73 of the holding unit 70 is preferably formed in a film shape to enable acoustic transmission. A polyethylene film for example may be used.

An acoustic matching medium is preferably provided between the bottom face 73 of the holding unit 70 and the living body 50 in order to suppress reflection of acoustic waves. For example, impedance matching gel can be used as the acoustic matching medium.

The holding unit 71 is preferably made of a material which is highly optically transmissive and has high durability against light. Glass or acrylic, for example, may be used as such a material.

The irradiation light, the probe, the signal processing unit, and the image display device used in this third embodiment may be the same as those of the first embodiment.

The use of the living body information imaging apparatus as described in this embodiment makes it possible to perform imaging of object information over a wide range in a depth direction in the object while suppressing deterioration of the image.

### Example 1

Example 1 shows a result of experiments conducted to study influence exerted on an image by changing the angle formed between the planar light irradiation surface of the object and the receiving element surface of the probe.

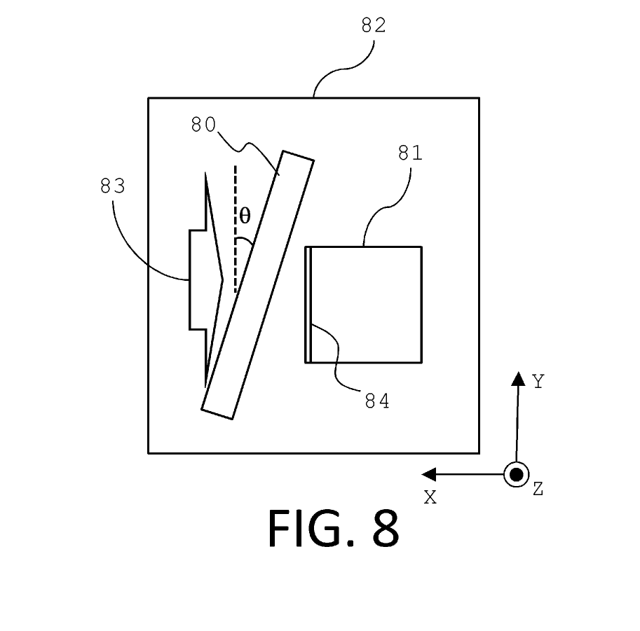

FIG. 8 shows an experimental system. A phantom 80 made of urethane having a thickness of 0.5 cm was used as the object. The phantom 80 had optical coefficients (μa, μs′) similar to those of the living body. The phantom 80 and the probe 81 are arranged within a water tank 82 which is filled with water.

As shown in FIG. 8, the phantom 80 is irradiated with light 83. Used as the light source was a YAG laser having a pulse width of 50 nanoseconds and a wavelength of 1064 nm. The irradiation light was applied to the phantom after being enlarged to a diameter of 6 cm.

Used as the probe 81 was an array transducer in which receiving elements were arranged two-dimensionally. The number of receiving elements was 15×23 elements. The receiving elements were each made of a PZT having a center frequency of 1 MHz, and had a shape of a square with each side measuring a slightly smaller than 2 mm.

Measurement was conducted while the angle θ between the planar light irradiation surface of the object and the receiving element surface 84 of the probe 81 is changed by rotating the phantom 80 with the probe 81 being fixed. Image reconstruction was performed using a photoacoustic signal obtained from each of the receiving elements. Universal back-projection was used for reconstruction.

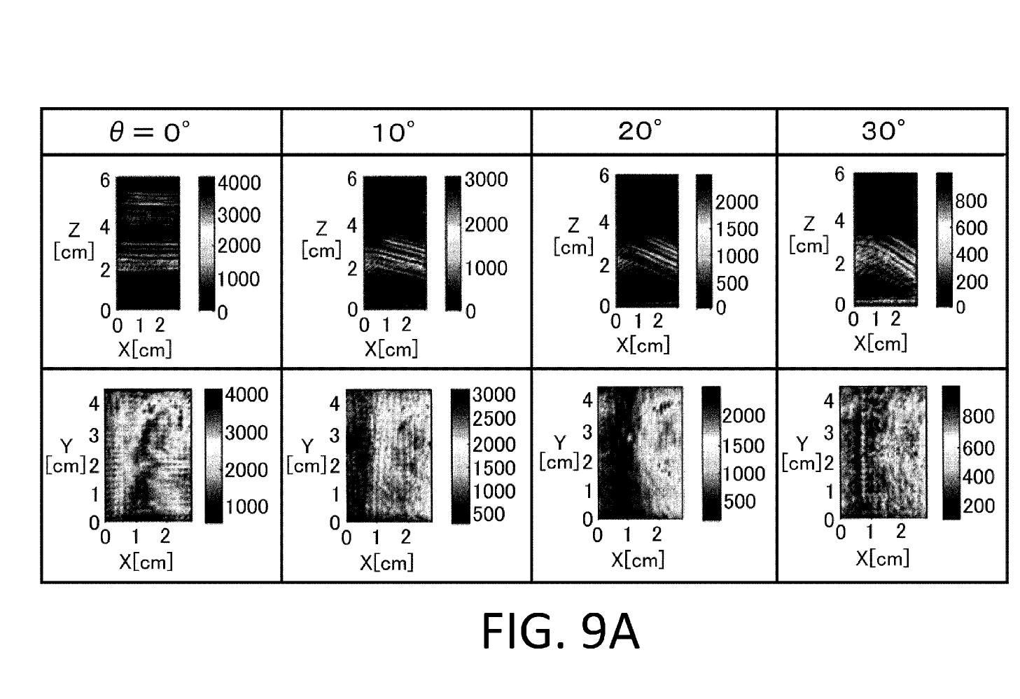

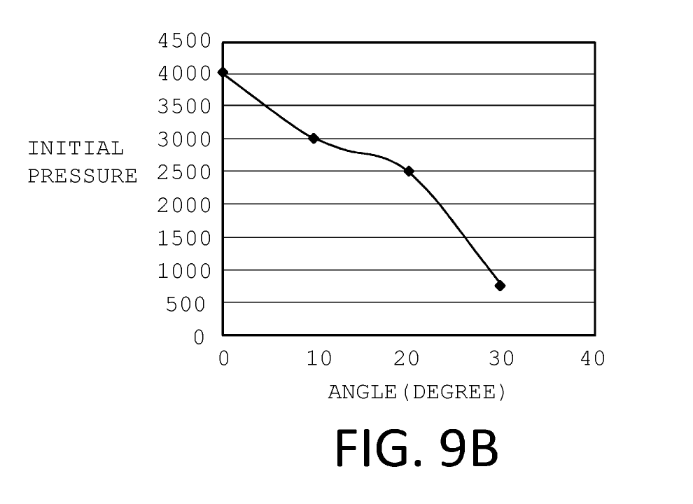

FIG. 9A shows reconstructed images using photoacoustic waves obtained when changing the angle θ.

FIG. 9B is a graph obtained by plotting intensity maximum values of the photoacoustic waves generated from a light irradiation plane of the phantom in FIG. 9A. The horizontal axis represents angle θ, and the vertical axis represents intensity (initial pressure). It can be seen that the intensity is decreased as the angle θ is increased from 0 degrees. When the angle θ was increased to 20 degrees, the intensity was decreased to about 60% of the one when the angle θ was 0 degrees. When the angle θ was further increased to 30 degrees, the intensity was decreased to about one fourth or less of the one when the angle θ was 0 degrees. It is presumed, as a reason why the intensity was not decreased so much as expected based on the expression (3) relating to directivity of reception of supersonic waves, that sufficient uniformity could not been achieved in the irradiation light 83 and the photoacoustic waves could not be obtained as perfect plane waves.

As seen from FIG. 9A, the maximum scale value become smaller as the angle θ is increased, which means that the influence of the photoacoustic waves generated from the light irradiation plane is decreased.

When an angle θ is formed between the planar light irradiation face of the object and the receiving element surface 84 of the probe, plane waves (photoacoustic waves) generated from the light irradiation face of the object are incident on the receiving element at an incident angle θ. Therefore, it is believed that the plane waves become difficult to receive due to the directivity of the probe when the angle θ is increased.

As described above, the influence exerted on the image by the photoacoustic waves generated from the light irradiation plane of the object was decreased by forming an angle between the planar light irradiation face of the object and the receiving element surface of the probe.

### Example 2

Example 2 will be described in terms of a configuration example in which the result of Example 1 is applied. The apparatus configuration of FIG. 6 is employed in this Example.

Used as the object 50 is a phantom made of urethane with a thickness of 5 cm. The phantom has optical coefficients (μa, μs′) similar to those of the living body. There exists in the urethane phantom, a spherical optical absorber 54 having an optical coefficient that is three times the optical coefficient μa. The holding unit 51 is made of polymethylpentene, while the holding unit 52 is made of acrylic. Used as the light source is a YAG laser having a pulse width of 50 nanoseconds and a wavelength of 1064 nm. The probe 57 is the same as the one used in Example 1.

The member 62 disposed between the living body 50 and the probe 57 has an angle of 20 degrees. The member 62 is made of polymethylpentene.

Matching gel is provided between the holding unit 51 and the living body 50, between the holding unit 51 and member 62, and between the probe 57 and member 62.

Image reconstruction is performed by universal back-projection using a photoacoustic signal obtained from each of the receiving elements. The universal back-projection is performed in consideration of change in index refraction of the acoustic waves or sonic speed of the phantom 50 and the holding unit 51 or the member 62, and the shape of the holding unit 51 or the member 62.

When the member 62 is provided with an angle θ of 30 degrees, compared with the case the member 62 is not provided, that is, the angle θ is 0 degrees, the intensity in the reconstructed image by photoacoustic waves generated from the light irradiation plane of the phantom is decreased to one fourth or less. On the other hand, the intensity in the reconstructed image by photoacoustic waves generated from the spherical optical absorber 54 remains at a similar value even when θ is 30 degrees in comparison when θ is 0 degrees.

Consequently, the configuration of Example 2 makes it possible to perform imaging of optical property value distribution over a wide range in a depth direction within the living body while suppressing deterioration of the image.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2011-001888, filed on Jan. 7, 2011, and Japanese Patent Application No. 2011-267794, filed on Dec. 7, 2011, which are hereby incorporated by reference herein in their entirety.

1.-14. (canceled)

15. A measuring apparatus comprising:
a holding unit configured to hold an object; and
a plurality of receiving elements configured to receive, through said holding unit, an acoustic wave generated by the object irradiated with light, wherein
said plurality of receiving elements are each respectively arranged such that a direction of a normal to an object surface held by said holding unit is non-parallel to a direction in which that receiving element exhibits the highest reception sensitivity.

16. A measuring apparatus comprising:
a holding unit configured to hold an object; and
a plurality of receiving elements configured to receive, through said holding unit, an acoustic wave generated by the object irradiated with light,
wherein said plurality of receiving elements are each respectively arranged such that a direction of a normal to an object surface at an intersection of a direction in which that receiving element exhibits the highest reception sensitivity and the object surface is non-parallel to the direction in which that receiving element exhibits the highest reception sensitivity.

17. A measuring apparatus comprising:
a holding unit configured to hold an object; and
a plurality of receiving elements configured to receive, through said holding unit, an acoustic wave generated by the object irradiated with light,
wherein said plurality of receiving elements are each respectively arranged such that an incident direction of the acoustic wave, which is generated from an object surface of the object held by said holding unit, to that receiving element is non-parallel to a direction in which that receiving element exhibits the highest reception sensitivity.

18. A measuring apparatus comprising:
a holding unit configured to hold an object; and
a plurality of receiving elements configured to receive, through said holding unit, an acoustic wave generated by the object irradiated with light,
wherein said plurality of receiving elements are each arranged such that a traveling direction of a plane wave which is generated in the vicinity of an object surface irradiated with light is non-parallel to a direction in which each of said receiving element exhibits the highest reception sensitivity.

19. A measuring apparatus comprising:
a holding unit configured to hold an object; and
a plurality of receiving elements configured to receive, through said holding unit, an acoustic wave generated by the object irradiated with light,
wherein said plurality of receiving elements are each respectively arranged such that a direction of a normal to a face opposite to a face of said holding unit on which that receiving element is arranged is non-parallel to a direction in which that receiving element exhibits the highest reception sensitivity.

20. A measuring apparatus comprising:
a holding unit configured to hold an object; and
a plurality of receiving elements configured to receive, through said holding unit, an acoustic wave generated by the object irradiated with light,
wherein said plurality of receiving elements are each respectively arranged such that a direction of a normal to a face opposite to a face of said holding unit on which that receiving element is arranged, at an intersection of the face opposite to said face of said holding unit on which that receiving element is arranged and a direction in which that receiving element exhibits the highest reception sensitivity is non-parallel to the direction in which that receiving element exhibits the highest reception sensitivity.

21. A measuring apparatus according to claim 15, wherein said plurality of receiving elements are each respectively arranged such that an angle between a direction of a normal to an object surface of the object held by said holding unit and a direction in which that receiving element exhibits the highest reception sensitivity is equal to or more than 10 degrees but not more than 80 degrees.

22. A measuring apparatus according to claim 15, wherein said plurality of receiving elements are each respectively arranged such that an angle between a direction of a normal to a face opposite to a face of said holding unit on which that receiving element is arranged and a direction in which that receiving element exhibits the highest reception sensitivity is equal to or more than 10 degrees and not more than 80 degrees.

23. A measuring apparatus according to claim 15, wherein said plurality of receiving elements are each respectively arranged such that a reception sensitivity at a direction of a normal to an object surface of the object held by said holding unit is equal to or less than one-fourth of a maximum value and not less than zero.

24. A measuring apparatus according to claim 15, wherein said plurality of receiving elements are each respectively arranged such that a reception sensitivity at a direction of a normal to a face opposite to a face of said holding unit on which that receiving element is arranged is equal to or less than one-fourth of a maximum value and not less than zero.

25. A measuring apparatus according to claim 15, wherein said plurality of receiving elements are each respectively arranged such that an angle between a direction of a normal to an object surface held by said holding unit and a direction in which that receiving element exhibits the highest reception sensitivity is adjusted according to an element size of that receiving element and a reception frequency.

26. A measuring apparatus according to claim 15, wherein said plurality of receiving elements are each respectively arranged such that an angle between a direction of a normal to a face opposite to a face of said holding unit on which that receiving element is arranged and a direction in which that receiving element exhibits the highest reception sensitivity is adjusted according to an element size of that receiving element and a reception frequency.

27. A measuring apparatus according to claim 15, wherein said holding unit is arranged such that only a portion of an object surface of the object held by said holding unit is irradiated with the light.

28. A measuring apparatus according to claim 15, wherein said plurality of receiving elements are arranged on a receiving element face.

29. A measuring apparatus according to claim 28, wherein said receiving element face is a plane.

30. A measuring apparatus according to claim 28, wherein said plurality of receiving elements are each respectively arranged such that said receiving element face of that receiving element is non-parallel to an object surface of the object held by said holding unit.

31. A measuring apparatus according to claim 28, wherein said plurality of receiving elements are each respectively arranged such that said receiving element face of that receiving element is non-parallel to a face opposite to a face of said holding unit on which that receiving element is arranged.

32. A measuring apparatus according to claim 15, wherein said holding unit comprises a pair of holding members holding the object therebetween.

33. A measuring apparatus according to claim 32, wherein said holding unit is arranged such that the object is irradiated with the light via one of said pair of holding members, on which said plurality of receiving elements are arranged.

34. A measuring apparatus according to claim 15, wherein
said holding member includes a holding face holding the object and a face non-parallel to said holding face; and
said plurality of receiving elements are each respectively arranged such that the direction in which that receiving element exhibits the highest reception sensitivity is perpendicular to said face non-parallel to said holding face.

35. A measuring apparatus according to claim 15, wherein said holding unit comprises a film-like member.

36. A measuring apparatus according to claim 15, wherein, for each of said receiving elements, the direction in which that receiving element exhibits the highest reception sensitivity is a direction normal to a surface of that receiving element.

37. A measuring apparatus according to claim 15, further comprising:
a signal processor configured to obtain a distribution of optical property values of the object or a distribution of density of substances forming the object based on electric signals output from respective ones of said plurality of receiving elements.

38. A measuring apparatus comprising:
a holding unit configured to hold an object; and
a receiving element configured to receive, through said holding unit, an acoustic wave generated by the object irradiated with light,
wherein said receiving element is arranged such that a direction of a normal to an object surface of the object held by said holding unit is non-parallel to a direction in which said receiving element exhibits the highest reception sensitivity.
